(19) United States
(12) Patent Application Publication
(10) Pub. No.: US 2014/0213849 A1
(43) Pub. Date: Jul. 31, 2014

(54) METHOD OF VIEWING INTERNAL ORGANS FROM DIFFERENT ANGLES

(71) Applicant: Covidien LP, Mansfield, MA (US)

(72) Inventor: Ashwini K. Pandey, Wallingford, CT (US)

(73) Assignee: Covidien LP, Mansfield, MA (US)

(21) Appl. No.: 14/150,443

(22) Filed: Jan. 8, 2014

Related U.S. Application Data
(60) Provisional application No. 61/756,651, filed on Jan. 25, 2013.

(51) Int. Cl. A61B 1/313; A61B 1/00; A61B 1/05

(52) U.S. Cl. CPC A61B 1/313; A61B 1/05; A61B 1/00071
USPC 600/109

(57) ABSTRACT

A laparoscope for introduction into a body cavity includes an elongated shaft having a proximal portion and a distal portion. Each of the proximal and distal portions has an outer surface. The distal portion is disposed in a bent configuration relative to the proximal portion once inserted into the body cavity. At least one illumination device is disposed on the outer surface of the distal portion and is in electronic communication with a control unit. At least one image capturing device is disposed adjacent the illumination device and is in electronic communication with the control unit and a display unit.

### CROSS-REFERENCE TO RELATED APPLICATIONS

The present application claims priority to, and the benefit of, U.S. Provisional Patent Application Ser. No. 61/756,651, filed on Jan. 25, 2013, the entire contents of which are hereby incorporated by reference.

### BACKGROUND

1. Technical field

The present disclosure relates to surgical instruments and particularly to laparoscopic instruments, including a shaft made from shape memory or super elastic alloy having an illumination device and an image capturing device thereon, which facilitate viewing internal orgasm from several different angles.

2. Background of Related Art

Laparoscopy, also called minimally invasive surgery (MIS), is a modern surgical technique in which operations in the abdomen are performed through small incisions as compared to larger incisions needed in traditional surgical procedures. Laparoscopy provides a number of advantages versus open procedures that include reduced pain from infection and hemorrhaging and shorter recovery time. The abdomen is usually insufflated, or essentially blown up like a balloon, with carbon dioxide gas (CO₂). This elevates the abdominal wall above the internal organs like a dome to create a working and viewing space. CO₂ is used because it is common to the human body and can be absorbed by tissue and removed by the respiratory system. It is also non-flammable, which is important because electrosurgical devices are commonly used in laparoscopic procedures.

Known laparoscopy technologies are limited in scope and complexity due in part to limited visual feedback. Using a single image from a typically rigid laparoscope inserted through a small incision limits the overall understanding of the surgical environment. Further, current technology requires a third port to accommodate a laparoscope (camera), and each new viewpoint requires an additional incision.

To obtain a different view, the surgeon has to move and adjust the laparoscope and this has to be done several times during a typical surgery. The present disclosure provides a system and method for inserting several low profile cameras inside the abdominal cavity that can be arranged as needed to provide views of internal organs from different angles.

### SUMMARY

In an embodiment of the present disclosure, a laparoscope is provided for insertion through a cannula suitable for introduction into a body cavity. The laparoscope includes an elongated shaft having a proximal portion and a distal portion. Each of the proximal and distal portions has an outer surface, the distal portion disposed in a bent configuration relative to the proximal portion once inserted into the body cavity. At least one illumination device is disposed on the outer surface of the distal portion, wherein the at least one illumination device is in electronic communication with a control unit. At least one image capturing device is disposed adjacent the illumination device, wherein the at least one illumination device is in electronic communication with the control unit and a display. Preferably, the image capturing device is a low profile camera.

Preferably, the elongated shaft is made from shape memory alloy and the elongated shaft is pre-bent prior to insertion through the cannula. A super elastic alloy may also be used. The elongated shaft is inserted into the body in a straight configuration and transitions to the bent configuration in response to temperature within the body cavity. Alternatively, the elongated shaft transitions to the bent configuration in response to an electrical signal from the control unit.

In another embodiment, a method is disclosed for providing a view of internal organs. The method includes the steps of providing a cannula inserted into a body exposing a body cavity. The method also includes providing at least one laparoscope, the laparoscope having an elongated shaft made from shape memory alloy, the elongated shaft having a proximal portion and a distal portion, each of the proximal and distal portions having an outer surface. At least one illumination device is disposed on the outer surface of the distal portion and at least one image capturing device is disposed adjacent the illumination device. The method further includes pre-bending the distal portion of the elongated shaft into the desired bent configuration relative to the proximal portion and releasing the distal portion such that the elongated shaft resumes a straight configuration. Next, the elongated shaft is advanced in the straight configuration through the cannula. Finally, the method includes actuating the illumination device and the image capturing device of the distal portion of the elongated shaft through a control unit and viewing internal organs through a display unit.

Accordingly to still another embodiment of the present disclosure, a kit may be provided for viewing internal organs from different angles. The kit has a control unit, a display unit, and a laparoscope. The laparoscope includes an elongated shaft, the elongated shaft having a proximal portion and a distal portion, each of the proximal and distal portions having an outer surface, the distal portion is disposed in a bent configuration relative to the proximal portion once inserted into a body cavity. At least one illumination device is disposed on the outer surface of the distal portion, wherein the at least one illumination device is in electronic communication with the control unit. At least one image capturing device is disposed adjacent the illumination device, wherein the at least one illumination device is in electronic communication with the display unit.

### BRIEF DESCRIPTION OF THE DRAWINGS

The above and other aspects, features, and advantages of the present disclosure will become more apparent in light of the following detailed description when taken in conjunction with the accompanying drawings in which:

FIG. 1 is a side view of an elongated shaft according to the system of the present disclosure;

FIG. 2 is a side view of a plurality of elongated shafts of FIG. 1 advanced through a cannula; and

FIG. 3 is a view of a plurality of elongated shafts deployed inside a body cavity according to an embodiment of the present disclosure.

### DETAILED DESCRIPTION OF THE EMBODIMENTS

Embodiments of the present disclosure will now be described in detail with reference to the drawings, in which like reference numerals designate identical or corresponding elements in each of the several views. As used herein, the term “distal,” as is conventional, will refer to that portion of the instrument, apparatus, device or component thereof which is farther from the user while, the term “proximal,” will refer to that portion of the instrument, apparatus, device or component thereof which is closer to the user. In the following description, well-known functions or constructions are not described in detail to avoid obscuring the present disclosure in unnecessary detail. Although the present disclosure is discussed in terms of a minimally invasive laparoscopic procedure, the presently disclosed instrument is usable in other minimally invasive procedures.

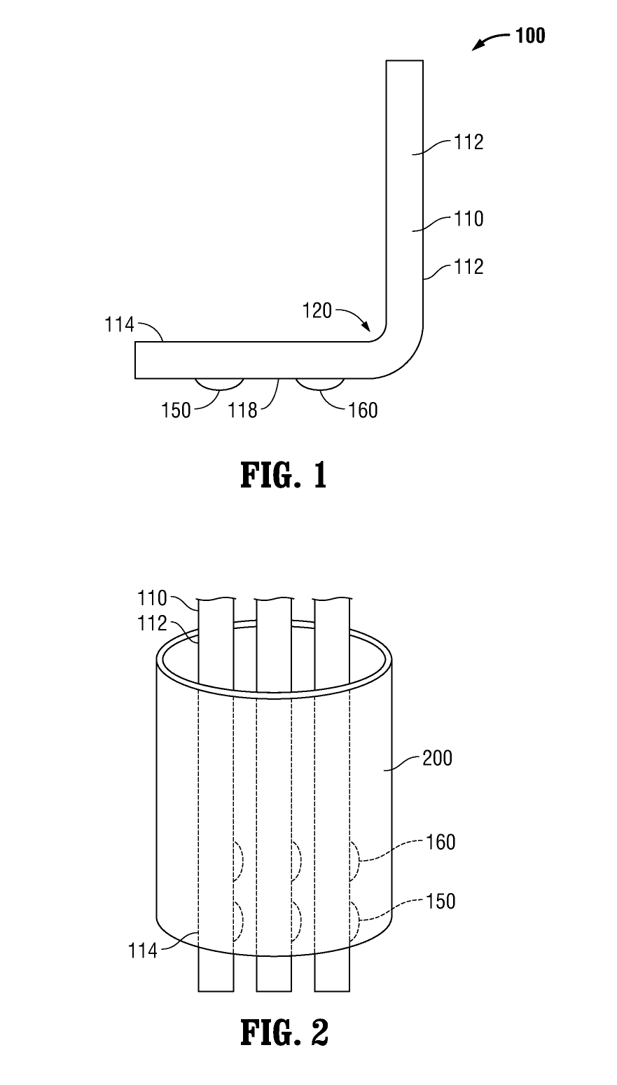

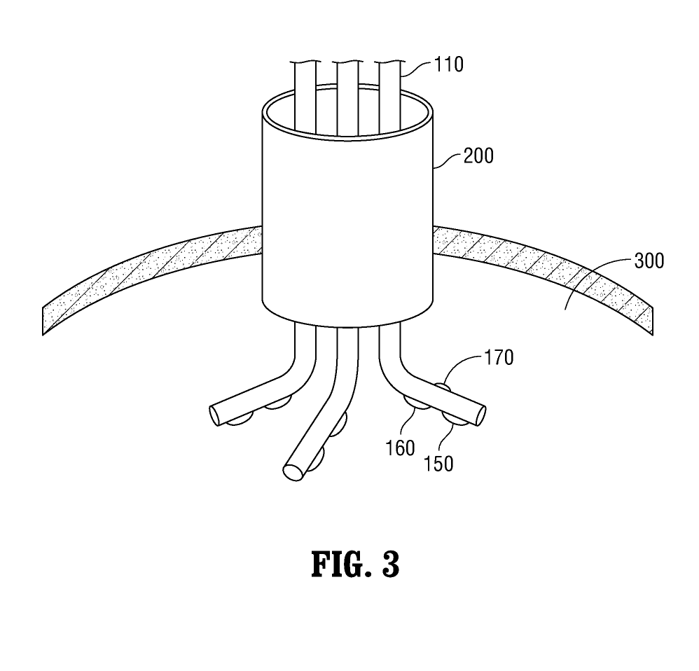

As shown in FIG. 1, an exemplary laparoscope 100 according to an embodiment of the present disclosure is illustrated. The laparoscope 100 includes an elongated shaft 110 having a proximal portion 112 and a distal portion 114. In one preferred embodiment, the elongated shaft 110 is made from shape-memory alloy (e.g. Nitinol) such that the elongate shaft 110 may have a straight or linear cylindrical configuration in a relaxed state (i.e., in the absence of externally applied forces). In the linear configuration, the distal portion 114 of the laparoscope 100 is inserted into a conventional trocar sleeve or cannula 200 (shown in FIG. 2), which is essentially a straight, hollow instrument that allows the laparoscope 100 to be inserted into the abdominal cavity 300 (shown in FIG. 3) of a patient. The cannula 200 is typical of known cannulas in the art and is made from polyvinyl chloride (PVC) or any other flexible material suitable for use in a body cavity or other medical applications.

Prior to insertion of the elongated shaft 110 into the cannula 200 and the abdominal cavity 300, the surgeon adjusts the elongated shaft 110 into a pre-bent configuration to obtain an optimal viewing window. The elongated shaft 110 is then returned to the original straight configuration, as shown in FIG. 2. Once inserted into the abdominal cavity, the elongated shaft assumes the pre-bent configuration. In one embodiment, the elongated shaft 110 assumes the pre-bent configuration in response to temperature within the body cavity. In an alternate embodiment, the elongated shaft 110 responds to an electrical signal from a control unit.

With continued reference to FIG. 1, the proximal and distal portions 112, 114 of the elongated shaft 110 each have an outer surface 116, 118, respectively. The outer surface 118 of the distal portion 114 includes at least one image capturing device 150 thereon. The image capturing device 150 allows the surgeon to clearly view the inside of the body cavity 300. Preferably, the image capturing device 150 is a low profile camera so that the image capturing device 150 does not obstruct the surgeon's tools during the procedure. At least one illumination device 160 is disposed adjacent the image capturing device 150 to illuminate the inside of the body cavity 300 and aide in viewing the internal organs with the image capturing device 150.

The image capturing device 150 and illumination device 160 can be disposed in varying configurations along the distal portion 114 of the elongated shaft 110. Based on the location of the image capturing device 150, the elongated shaft 110 is pre-bent to provide the desired location as well as optimal angle for the surgeon. This variation in the pre-bent configuration is determined by varying the length of the bent portion (i.e. the distal portion) and the angle of the bend 120. FIG. 1 illustrates the angle of the bend 12 as a generally ninety degree angle with the distal portion 114 being generally the same length as the proximal portion 112, however, it is understood that any bend angle to allow the surgeon to view the body cavity can be achieved. Further, FIG. 1 illustrates the image capturing device 150 and the illumination device 160 generally along the same longitudinal axis with the image capturing device 150 being near a distal end of the elongated shaft 110. It will be appreciated that this configuration can be altered during manufacturing based on the particular needs of the surgical procedure.

FIG. 2 illustrates several laparoscopes 100 introduced into the cannula 200 and suitable for introduction into the body cavity 300. Cannula 200 shown in FIG. 2 is shown has a straight cylindrical hollow body, however, it will be appreciated that varying type of cannulas well known in the art may be utilized. For example, cannulas having multiple longitudinal holes extending therethrough to accept a laparoscope in each hole may be utilized and cannulas of varying shapes and sized depending the surgical procedure and/or patient may be used. As shown in FIG. 2, once the cannula 200 is introduced into the body cavity 300, the elongated shaft 110 in the original straight configuration and is advanced through the cannula 200 to be introduced into the body cavity 300.

As shown in FIG. 3, once the individual laparoscopes are positioned through the cannula and advanced into the body cavity, the elongated shafts 110 assume their pre-bent shape. As noted above, the shape memory allow of the elongated shaft 110 allows the laparoscope 100 to assume the pre-bent configuration based on the response to temperature within the body cavity.

Alternatively, the elongated shaft may respond to an electrical signal from a control unit (not shown), operated by the surgeon. In this embodiment, at least one electrode 170 is disposed along the outer surface 118 of the distal portion 114. The electrode 170 is in communication with a generator (not shown) of the control unit. Once the surgeon has advanced the laparoscope 100 into the abdominal cavity 300, the surgeon activates the generator to send a signal to the electrode 170. The electrode 170 heats at least a portion of the distal portion 114 of the elongated shaft 100 to assume the pre-bent configuration. .This change in shape may also be caused by the heat within the body of the patient.

After the elongated shaft 110 has assumed the pre-bent configuration, the surgeon positions each individual laparoscope 100 as desired by maneuvering the proximal portion 112 of the elongated shaft 110 that is generally outside the abdominal cavity 300. The surgeon views the inside of the abdominal cavity 300 using the image capturing devices and positions each laparoscope 100 such that the entire section of the abdominal cavity 300 is visible for the surgical procedure. Once the surgeon is satisfied with the positioning and views provided by each image capturing device, the individual laparoscopes are locked into place within the body cavity in the their pre-bent configurations. The surgeon then completes the procedures using the image capturing devices as a guide for viewing the internal organs from different angles.

While several embodiments of the disclosure have been shown in the drawings, it is not intended that the disclosure be limited thereto, as it is intended that the disclosures be as broad in scope as the art will allow and that the specification be read likewise. Therefore, the above description should not be construed as limiting, but merely as exemplifications of particular embodiments.

What is claimed is:

1. A laparoscope for insertion through a cannula suitable for introduction into a body cavity, the laparoscope comprising:
an elongated shaft, the elongated shaft having a proximal portion and a distal portion, each of the proximal and distal portions having an outer surface, the distal portion disposed in a bent configuration relative to the proximal portion once inserted into the body cavity;
at least one illumination device disposed on the outer surface of the distal portion, wherein the at least one illumination device is in electronic communication with a control unit; and
at least one image capturing device disposed adjacent the illumination device, wherein the at least one image capturing device is in electronic communication with the control unit and a display unit.

2. The laparoscope of claim 1, wherein the elongated shaft is made from shape memory alloy.

3. The laparoscope of claim 2, wherein the elongated shaft is pre-bent prior to insertion through the cannula.

4. The laparoscope of claim 1, wherein the image capturing device is a low profile camera.

5. The laparoscope of claim 4, wherein the elongated shaft is inserted into the cannula in a straight configuration and transitions to the bent configuration in response to temperature within the body cavity.

6. The laparoscope of claim 4, wherein is inserted into the body in a straight configuration and transitions to the bent configuration in response to an electrical signal from the control unit.

7. A method for providing a view of internal organs, the method comprising:
providing a cannula inserted into a body exposing an abdominal cavity;
providing at least one laparoscope, the laparoscope comprising:
an elongated shaft made from shape memory alloy, the elongated shaft having a proximal portion and a distal portion, each of the proximal and distal portions having an outer surface;
at least one illumination device disposed on the outer surface of the distal portion; and
at least one image capturing device disposed adjacent the illumination device;
pre-bending the distal portion of the elongated shaft into a desired bent configuration relative to the proximal portion;
releasing the distal portion such that the elongated shaft resumes a straight configuration;
advancing the elongated shaft in the straight configuration through the cannula such that distal portion elongated shaft assumes the desired pre-bent configuration once within the abdominal cavity;
actuating the illumination device and the image capturing device through a control unit; and
viewing internal organs through a display unit.

8. The method of claim 7, wherein the elongated shaft is made from shape memory alloy.

9. The method of claim 8, wherein the image capturing device is a low profile camera.

10. The method of claim 9, further comprising the step of transitioning the elongated shaft from a straight configuration to the bent configuration in response to temperature within the body cavity.

11. The method of claim 9, further comprising the step of transitioning the elongated shaft from a straight configuration to the bent configuration in response to an electrical signal from the control unit.

12. The method of claim 9, further comprising the step of cooling the elongated shaft below ambient temperature prior to transitioning from a straight configuration to the bent configuration.

13. A kit for viewing internal organs from different angles, the kit comprising:
a control unit;
a display unit; and
a laparoscope, the laparoscope including:
an elongated shaft, the elongated shaft having a proximal portion and a distal portion, each of the proximal and distal portions having an outer surface, the distal portion disposed in an bent configuration relative to proximal portion once inserted into a body cavity;
at least one illumination device disposed on the outer surface of the distal portion, wherein the at least one illumination device is in electronic communication with the control unit; and
at least one image capturing device disposed adjacent the illumination device, wherein the at least one illumination device is in electronic communication with the display unit.

14. The kit of claim 13, wherein the elongated shaft is made from shape memory alloy.

15. The kit of claim 14, wherein the elongated shaft is pre-bent prior to insertion through the cannula.

16. The kit of claim 15, wherein the image capturing device is a low profile camera.

17. The kit of claim 16, wherein the elongated shaft is inserted into the body in a straight configuration and transitions to the bent configuration in response to temperature within the body cavity.

18. The kit of claim 17, wherein the elongated shaft is cooled below ambient temperature prior to being inserted into the body.

19. The kit of claim 17, wherein the elongated shaft is inserted into the body in a straight configuration and transitions to the bent configuration in response to an electrical signal from the control unit.
